(19) United States
(12) Patent Application Publication
(10) Pub. No.: US 2009/0030219 A1
(43) Pub. Date: Jan. 29, 2009

(54) HETEROGENEOUS ACID-CATALYZED PROCESS FOR BIODIESEL PRODUCTION FROM FATTY ACIDS

(75) Inventor: Chia-Hung Su, Taipei County (TW)

Correspondence Address: WPAT, PC;INTELLECTUAL PROPERTY ATTORNEYS, 2030 MAIN STREET, SUITE 1300, IRVINE, CA, 92614, US

(73) Assignees: Mon-Han Wu, Tainan (TW); SUSTINEO BIOTECHNOLOGY CO., LTD., Tainan (TW)

(21) Appl. No.: 11/782,431

(22) Filed: Jul. 24, 2007

(51) Int. Cl. C07C 51/00

(52) U.S. Cl. 554/124

(57) ABSTRACT

The present invention relates to an acid-catalyzed mixture comprising (a) free fatty acids; (b) alcohol; and (c) acid exchange resin. The present invention also relates to a method for preparing a biodiesel.

### BACKGROUND OF THE INVENTION

1. Field of the Invention

This invention relates to an improved composition containing normal oil or fat including free fatty acid, methanol and acidic cation-exchange resin as a heterogeneous catalyst and a process to producing a biodiesel and the product thereof.

2. Description of the Related Art

Environment problems coupled with petroleum reserve depletion stimulated research to develop the alternative sources. Biodisel is one of the candidates, which has similar combustion properties as diesel and has almost no sulphur, no aromatics and 10% built-in oxygen, which helps it to burn well therefore it is being used to reduce the air pollution. Besides, developing biodiesel is benefit to agriculture and to economic stability due to reduction of the use of the fossil fuel, a limited resources localized to some regions.

Fatty acids methyl esters have properties similar to petroleum diesel and are regarded as biofuel or biodiesel. Biodiesel refers to alkyl esters made from transesterification of virgin or used plant oils or animal fats with the short chains alcohol. This biomass fuel has received much attention, since it is a kind of alternative, biodegradable, non-toxic and renewable energy. In addition, biodiesel does not contribute to the net carbon dioxide in the atmosphere, because it is regenerated by photosynthesis. Pure biodiesel is available at many gas stations in Germany.

For industrial biodiesel production, homogeneous basic catalysts such as sodium or potassium methoxide and hydroxide, are commonly used for transesterification of oil and methanol to produce fatty acid methyl esters and glycerol. However, the undesired side reaction of saponification occurs since the added catalyst reacts with free fatty acids (FFAs) present in unrefined oil. In order to solve the side reaction problem of saponification, homogeneous acidic catalysts such as hydrochloric acid or sulfuric acid, are used to esterify the FFAs and methanol into fatty acid methyl esters. However, processes dependent on homogeneous acidic or basic catalysts, require large amounts of water to remove the catalyst. The waste water of washed chemicals leads to serious contamination and pollution problems

Enzyme based processes can circumvent above problems and are attractive alternatives. For example, the enzyme lipases can transesterify triglyceride and methanol to fatty acid methyl esters. Although the biochemical processes are expected to be highly selective and pollution free, process economic issues arising from high cost of enzyme and decay in enzymatic activity need to be addressed.

Recently, resins have been introduced as heterogeneous catalysts for biodiesel synthesis to solve the problem of chemical pollution. Anion-exchange resin was used as heterogeneous basic catalyst in the transesterification reaction of triolein with ethanol by optimal batch and continuous modes.

U.S. Pat. Application Ser. No. 2005/528333 to Connemann et al. disclosures a non-pressurized method for the continuous production of biodiesel made from biogenic fat- or oil-containing starting mixture in which the homogeneous catalyst need a lot of water to remove it.

U.S. Pat. Application Ser. No. 2006/077162 to Sharma et al. disclosures a single pot process performing esterification of non-edible oil containing free fatty acids by sulphuric acid as an acidic catalyst in a reaction vessel, attached with a column or soxhlet apparatus filled with a water adsorbent.

U.S. Pat. Application Ser. No. 2005/867627 of Portnoff et al. disclosures a process of converting feedstock into a biodiesel by utilizing one kind of a zeolite in the acid form as a heterogeneous acidic catalyst and applying additional energy such as microwave to improve the reaction rate.

### BRIEF DESCRIPTION OF THE DRAWINGS

FIG. 1 shows the conversion of FFAs versus time for fatty acid methyl esters formation (T=333.15 K, θ=10:1) catalyzed by different amounts of Dowex Monosphere 88 expressed as mass fraction to FFAs (▪, 53.6%; , 26.8%; ♦, 13.4%; ▴, 7.31%; ▾, 3.65%). The solid line represents the results of the pseudo-homogeneous model.

FIG. 2 demonstrates the effect f catalyst loading on reaction conversion and kinetic parameters (▪, Equilibrium conversion; , Equilibrium constant; ♦, Forward reaction rate constant).

FIG. 3 is the conversion of FFAs versus time for fatty acid methyl esters formation (catalyst loading=26.8% (W/W), initial reactant molar ratio of methanol to FFAs=10:1) at different reaction temperature (▪, 353.15 K; , 343.15 K; ♦, 333.15 K). The solid line represents the results of the pseudo-homogeneous model.

FIG. 4 depicts the conversion of FFAs versus time for fatty acids methyl esters formation (T=343.15K, catalyst loading=26.8% (W/W)) at different initial reactant molar ratio. (▪, 20:1; , 10:1; ♦, 1:1). The solid line represents the results of the pseudo-homogeneous model.

### SUMMARY OF THE INVENTION

The present invention provides an acid-catalyzed mixture comprising (a) free fatty acids; (b) alcohol; and (c) acid exchange resin.

The present invention also provides a method for producing a biodiesel comprising:
a) providing the mixture of the present invention; and
b) reacting the alcohol with free fatty acids in the presence of the acid exchange resin to obtain a mixture of fatty acid alkyl esters and triglycerol.

### DETAILED DESCRIPTION OF THE INVENTION

As used herein, the term “oil or fat” may refer to lipid derived from plant or animal sources in purified or unpurified form selected from the group consist of soapstock, brown grease, yellow grease, industrial tallow, industrial lard, animal fat waste products, edible tallow, unpurified crude vegetable oils and unpurified animal fats.

As used herein, the term “alcohol” is meant to refer to a hydrocarbon compound containing one or more hydroxyl groups such as methanol or ethanol. In the preferred embodiment, the alcohol is methanol or ethanol. In the more preferred embodiment, the alcohol is methanol.

As used herein, the term “free fatty acids” is meant to refer to an organic acids synthesized in nature by both animals and plants and containing a hydrocarbon group with 14 to 24 carbon atoms, possibly in a straight chain, although chains of 4 to 28 carbons may be found. Longer chains exist, but typically in low concentrations. Fatty acids are used to describe fatty acids that are not bound in ester compound. In the preferred embodiment, free fatty acids are prepared by hydrolysis of oils or fats, with impurity removed and confirmed the purity of free fatty acid. In the preferred embodiment, the hydrolysis is performed by lipase.

As used herein, the term “heterogeneous catalyst” may refer to a catalyst that is in a different phase as the reactants.

Accordingly, the present invention relates to an acid-catalyzed mixture comprising (a) free fatty acids; (b) alcohol; and (c) acid exchange resin.

In the mixture of the present invention, the resin is acid cation-exchange resin or acid anion-exchange resin. In the preferred embodiment, the resin is acid cation-exchange resin.

The present invention also provides a method for producing biodiesel and reacting alcohol with free fatty acids in the presence of an acid cation-exchange resin as a catalyst to obtain a mixture of fatty acid alkyl esters as a biodiesel in an economic and an environment- protected way.

Accordingly, the present invention also provides to a method for producing a biodiesel comprising:
a) providing the mixture of the present invention; and
b) reacting the alcohol with free fatty acids in the presence of the acid exchange resin to obtain a mixture of fatty acid alkyl esters and triglycerol.

The method of the present invention further comprises analyzing the free fatty acids conversion during esterification reaction by the use of acid-base titration.

### EXAMPLE

Fatty acid initial mixture is obtained from hydrolysis of soybean oil by mixing with water at room temperature. The initial molar ratio of water to oil is 6:1. The reaction was initiated by addition of enzyme solution (50 mg free lipase per milliliter of DI water) and the reaction was allowed to run overnight. In this way, FFAs were derived from the enzymatic hydrolysis of soybean oil. The supernatant containing high level of FFAs is harvested by centrifugation at 10000 rpm for 10 min at room temperature. The purity of FFAs in this supernatant is determined and it is used as the feedstock for the investigation of biodiesel production by the solid catalyzed esterification process.

The commercial acid resin, whose physical properties were illustrated as in Table 1 below, was initiated for esterification reaction by the following steps. First, the acidic resin was washed three times by DI water to remove the impurities. In order to excite the activities of Brönsted acid sites, the resin was then immersed in 1 M hydrochloric acid solution and agitated continuously for 1 hour followed by washing with DI water until neutral pH. The resin was recovered and dried overnight at 60° C. in an oven. The total amount of Brönsted acid sites was determined by acid-base titration methods. An amount of 1 g of resin was mixed with 200 ml of 0.1 M NaOH prepared in 5% NaCl, and allowed to stand overnight at room temperature. Fifty milliliter of the supernatant liquid was subsequently titrated with 0.1 M HCl using phenolphthalein as an indicator to determine residual amount of base (i.e. acidity of the resin).

TABLE 1

| Product name and provider | Dowex Monosphere 88 by Dow |
|---|---|
| | Chemical Company |
| Matrix | Styrene-divinylbenzene |
| Structure | Macroporous |
| Functional group | Sulfonate |
| Particle density [g L⁻¹] | 1.2 |
| Particle size [mm] | 0.5-0.6 |
| Acidity [mmol g⁻¹] | 4.48 |

For understanding the reaction kinetics of the esterification, three experimental parameters were considered. These were the fraction of resin weight to reactant, reaction temperature and the molar ratio of methanol to FFAs. The parametric effected with different levels were expressed as operating conditions of a batch reactor and summarized in Table 2.

TABLE 2

| | Catalyst loading | Temperature | Molar ratio of |
|---|---|---|---|
| Run | (weight of resin to FFAs, %) | (K) | methanol to FFAs |
| 1 | 3.65 | 333.15 | 10:1 |
| 2 | 7.31 | 333.15 | 10:1 |
| 3 | 13.4 | 333.15 | 10:1 |
| 4 | 26.8 | 333.15 | 10:1 |
| 5 | 53.6 | 333.15 | 10:1 |
| 6 | 26.8 | 343.15 | 10:1 |
| 7 | 26.8 | 353.15 | 10:1 |
| 8 | 26.8 | 343.15 | 1:1 |
| 9 | 26.8 | 343.15 | 20:1 |

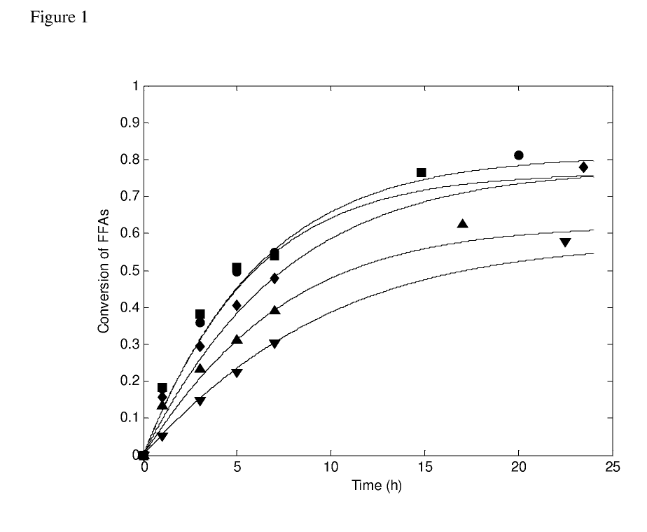

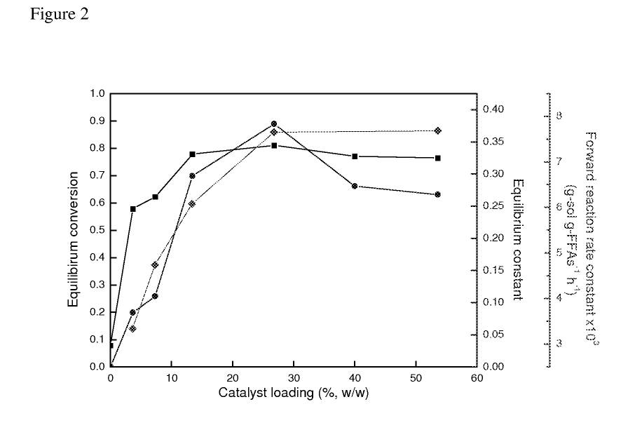

The catalyst loading was varied from 3.65 to 53.6% (w/w) of FFAs to evaluate its effect on the conversion of FFAs at the given temperature of 333.15 K and methanol:FFAs molar ratio of 10:1 (run 1 to 5 in Table 2). The time courses of FFAs conversion were shown in FIG. 1. It was observed that the higher the catalyst loading, the faster the rate was obtained because of the increase in the total number of active sites available for reaction. As illustrated in FIG. 1, the FFAs conversion increased with an increase in catalyst loading from 3.65 to 26.8% (w/w). The equilibrium conversion, equilibrium constant and forward reaction rate constant were dependent of catalyst loading (FIG. 2). The equilibrium conversion, equilibrium constant and forward reaction rate constant increased with an increase in catalyst loading varied from 0 to 26.8% (w/w). However, the reaction rate and final equilibrium conversion reached an upper limit when the loading (weight fraction) of catalytic resin exceeded 26.8% (w/w) (FIG. 2). At 26.8% (w/w) of catalyst loading used, the saturated equilibrium conversion and forward reaction rate constant was observed, and the maximum equilibrium constant was obtained. Hence, it could be concluded that the optimum catalyst loading was 26.8% (w/w).

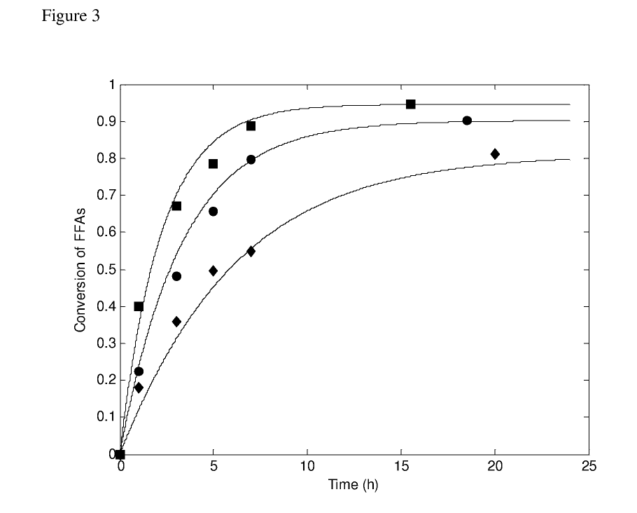

The study of temperature effect was very important for evaluating activation energy and intrinsic rate. The effect of reaction temperature was investigated in the range of 333.15 K to 353.15 K. The optimum catalyst loading (26.8%, w/w) and molar ratio of methanol to FFAs (θ=10) were kept constant for these experimental runs (run 4, 6 and 7 in Table 2). The time courses of FFAs conversion were displayed in FIG. 3. FIG. 3 indicated that the reaction rates and final conversion increases with increase in temperature at the optimal catalyst loading of 26.8% (w/w). In many esterification reactions, the heat of reaction was negligible and resulted in the equilibrium conversion being independent of temperature. However, the equilibrium conversion was observed to be dependent on temperature in this study. The equilibrium conversion increased from about 0.8 to 0.95 with an increase in temperature from 333.15 K to 353.15 K.

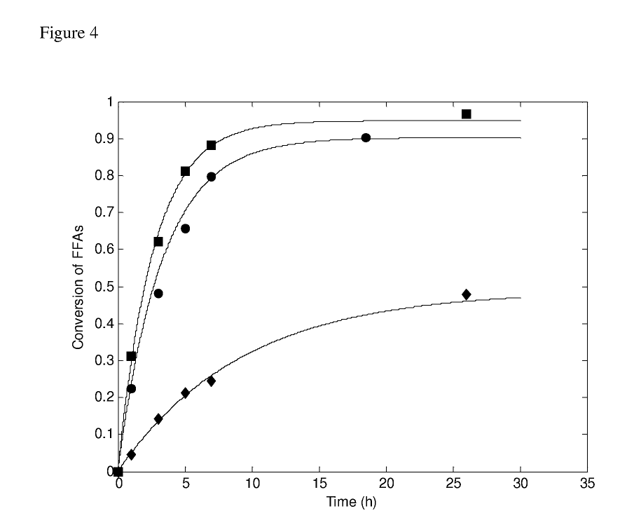

The initial molar ratio of methanol to FFAs was varied from 1:1 to 20:1 at a temperature of 343.15 K and 26.8% (w/w) catalyst loading (run 8, 6 and 9 in Table 2). FIG. 4 showed the experimental results. As observed, not only the reaction rate but also equilibrium conversion increased with the initial reactant molar ratio. The equilibrium conversion of FFAs increased from about 0.45 at a feed molar ratio (methanol to FFAs) of 1:1 to 0.96 at a feed molar ratio (methanol to FFAs) of 20:1.

Theoretically, the esterification of 1 mole of FFAs required 1 mole alcohol for yield of fatty acid methyl esters. According to LeChatelier's Principle, excess of alcohol used shifts the equilibrium of reversible reaction towards the direction of esters formation. In this work, increase in the methanol/FFAs molar ratio from 1:1 to 10:1 exhibited a significant effect on the fatty acid methyl esters formation. However, slightly significant effect on esters formation was observed when the initial molar ratio was further increased from 10:1 to 20:1. Excess methanol used in biodiesel production can be recycled to improve operation economics.

What is claimed is:

1. An acid-catalyzed mixture comprising (a) free fatty acids; (b) alcohol; and (c) acid exchange resin.

2. The mixture of claim 1 wherein the resin is acid cation-exchange resin.

3. The mixture of claim 1 wherein the (a) is prepared by hydrolysis of oils or fats.

4. The mixture of claim 1 wherein the (a) is natural occurring in the raw material obtained.

5. The mixture of claim 1 wherein the (b) is methanol.

6. The mixture of claim 1 wherein the (b) is ethanol.

7. The mixture of claim 3 wherein the hydrolysis is carried out by lipase.

8. A method for producing a biodiesel comprising:
a) providing the mixture of claim 1; and
b) reacting the alcohol with free fatty acids in the presence of the acid exchange resin to obtain a mixture of fatty acid alkyl esters and triglycerol.

9. The method of claim 8 further comprising analyzing the free fatty acids conversion during esterification reaction by using acid-base titration.

10. The method of claim 8 wherein the resin is acid cation-exchange resin.

11. The method of claim 8 wherein the alcohol is methanol.

12. The method of claim 8 wherein the alcohol is ethanol.
